(19) United States
(12) Patent Application Publication
(10) Pub. No.: US 2013/0034906 A1
(43) Pub. Date: Feb. 7, 2013

(54) METHODS FOR IN VITRO OOCYTE MATURATION

(71) Applicants: Gary D. Smith, Ann Arbor, MI (US); Shuichi Takayama, Ann Arbor, MI (US); Charles Bormann, Ann Arbor, MI (US); Yunseok Heo, Ann Arbor, MI (US)

(72) Inventors: Gary D. Smith, Ann Arbor, MI (US); Shuichi Takayama, Ann Arbor, MI (US); Charles Bormann, Ann Arbor, MI (US); Yunseok Heo, Ann Arbor, MI (US)

(73) Assignee: THE REGENTS OF THE UNIVERSITY OF MICHIGAN, Ann Arbor, MI (US)

(21) Appl. No.: 13/520,645

(22) PCT Filed: Jan. 7, 2011
(86) PCT No.: PCT/US11/20455

§ 371 Date: Oct. 24, 2012

Related U.S. Application Data
(60) Provisional application No. 61/292,994, filed on Jan. 7, 2010.

(51) Int. Cl. C12N 5/075; C12N 15/06

(52) U.S. Cl. 435/449; 435/395; 435/396; 435/373

(57) ABSTRACT

The invention relates to methods for assisted reproduction technology in mammals. Specifically the invention relates to methods for in vitro mammalian oocyte culture and oocyte maturation, in vitro fertilization, and in vitro embryo development.

This application claims priority to provisional application 61/292,994, filed Jan. 7, 2010, which is herein incorporated by reference in its entirety.

This invention was made with government support under contract number USDA; 2005-35203-16148 awarded by the U.S. Department of Agriculture. The government has certain rights in the invention.

### FIELD OF THE INVENTION

The invention relates to methods for assisted reproduction technology in mammals. Specifically the invention relates to methods for in vitro mammalian oocyte culture and oocyte maturation, in vitro fertilization, and in vitro embryo development.

### BACKGROUND OF THE INVENTION

In vitro embryo production (IVP) holds many benefits in agricultural livestock settings, facilitating the mass production of embryos at low cost. Using IVP techniques, embryos can be sexed and genetically analyzed (e.g., for diseases) prior to transfer to a donor. In addition, IVP allows clonal production of livestock lines bearing desirable qualities. IVP also facilitates embryo banking via cryopreservation. Additionally, IVP is used in human assisted reproduction technologies.

However, the IVP process suffers from reduced efficiency at multiple points. IVP involves oocyte maturation, in vitro fertilization, and in vitro embryo development. Sufficiently developed embryos (e.g., to blastocyst stage) are thereafter transferred to donors. Reduced efficiency and loss of oocyte or embryo viability can occur at any of these stages. In particular, in vitro oocyte maturation can contribute to a loss of developmental competence. Better methods for in vitro oocyte maturation would be of great benefit in human assisted reproduction techniques, particularly for patients who have not had success with conventional in vitro fertilization techniques (e.g., patients with ovarian hyperstimulation syndrome, patients that do not respond to gonadotropin stimulation). While improvements in culture media used for IVP in recent years has resulted in higher success rates, additional methods are needed to optimize efficiency of IVP, and particularly in vitro oocyte maturation.

### SUMMARY OF THE INVENTION

In certain embodiments, the invention relates to methods for assisted reproduction technology in mammals. In some embodiments, the invention relates to methods for in vitro mammalian oocyte culture and oocyte maturation, in vitro fertilization, and in vitro embryo development.

In experiments conducted during the course of developing some embodiments of the present invention, methods were developed and tested that provided continual refreshment of soluble environment during in vitro oocyte maturation of mammalian oocytes. While the present invention is not limited to any particular mechanism, and an understanding of the mechanism is not necessary to practice the present invention, it is contemplated that in a static soluble environment the high metabolic activity of non-gamete cells (cumulus cells) that accompany the immature oocyte produce waste products and/or over-use substrates, thus compromising oocyte health and subsequent embryonic developmental competence. While cumulus cells are important in cellular cross-talk with oocytes during times of nuclear maturation, they can at the same time be detrimental to subsequent normal function of the oocyte in a static soluble environment. Thus, while the present invention is not limited to any particular mechanism, and an understanding of the mechanism is not necessary to practice the present invention, it is contemplated that a continual dynamic culture condition enables maintenance of cumulus-oocyte cellular interactions and stimulatory cross-talk while providing renewable substrates for cumulus metabolism and removing metabolic waste produce by cumulus cells. In some embodiments, a pumping or reverse-exchange dynamic soluble environment is advantageous as oocytes mature and cumulus cells expand and subsequently have the potential of “clogging” flow-channels. In some embodiments, this pumping or reverse-exchange flow is preferential to uninterrupted flow that, once clogged, would lose the refresh soluble environment exchange. In some embodiments, pumping nature of the flow provides a developmental advantage. While the present invention is not limited to any particular mechanism, and an understanding of the mechanism is not necessary to practice the present invention, it is contemplated that these advantages impact oocyte cytoplasmic maturation and facilitate improved subsequent embryonic developmental competence. A continually refreshed soluble environment provides stable soluble medium composition in comparison to non-refreshed methods. Experiments conducted during the course of developing some embodiments of the present invention showed that significantly more blastocysts were produced from oocytes matured under dynamic flow (e.g., dynamic microfluidic) conditions than from oocytes matured under static conditions, indicating higher rates of developmental competence.

Methods of the present invention are not limited by physical aspects of the dynamic flow process. Dynamic flow of culture media may be achieved by passive or active means. In some embodiments, dynamic flow is achieved by peristaltic pumping. In some embodiments, dynamic flow is applied unidirectionally. In some embodiments, dynamic flow is applied in reverse-exchange mode. In some embodiments, dynamic flow is pulsatile. In some embodiments, dynamic flow is continuous (e.g., continuous perfusion). Methods of the present invention are not limited by rate of dynamic flow. In some embodiments where dynamic flow is achieved using peristaltic pumping, flow rate may be determined by setting pumping motion to 0.01 Hz, 0.01-0.1 Hz, 0.1-0.2 Hz, 0.2-0.4 Hz, 0.4-0.6 Hz, 0.6-0.8 Hz, 0.8-1.0 Hz, 1.0 Hz or above. In some embodiments, average flow rate may be 1-5 nL/min, 5-10 nL/min, 10-20 nL/min, 20-30 nL/min, 30-50 nL/min, 50 nL/min or more.

In certain methods of the present invention, stationary supports are utilized (e.g., to contact an oocyte; an embryo; an oocyte for in vitro maturation). In some preferred embodiments, a microfluidic chip comprises a stationary support. Methods of the present invention are not limited by physical dimensions, structural features, or materials used in the manufacture of the microfluidic chip. In some embodiments, the microfluidic chip comprises a layer of poly-dimethylsiloxane (PDMS). In some embodiments, the PDMS layer bears structural features (e.g., channels, microfunnels, depressions). In some embodiments, the PDMS layer contacts a poly-dimethylsiloxane-parylene-poly-dimethylsiloxane hybrid membrane. In some embodiments, the contacting is direct. In some embodiments, the contacting is indirect.

In some embodiments, methods of the present invention utilize devices designed to provide pulsatile dynamic flow. In some embodiments, such devices are computer-controlled. In some embodiments, such devices utilize piezoelectric, moveable pins. In some embodiments, such pins comprise a custom Braille display. Examples of devices compatible with methods of the present invention include but are not limited to those described in Futai et al. (2006) Lab Chip 6:149-154; Kamotani et al. (2008) Biomaterials 29:2646-2655; and PCT Patent App. No. PCT/US09/55722; each herein incorporated by reference in its entirety.

Methods of the present invention find use with mammalian species. Examples of mammalian species include rodents, human, non-human primates, equines, canines, felines, bovines, porcines, ovines, lagomorphs, and the like.

Methods of the present invention are not limited by duration of culturing steps. Each step of culturing methods described herein (e.g., in vitro embryo maturation, in vitro fertilization, in vitro embryo development) may be conducted for less than 1 hour, 1-12 h, 12-24 h, 24-36 h, 36-48 h, 48 h-3 days, 3-6 days, 6-10 days, 10-20 days, 20 days or more.

In certain embodiments, the present invention provides a method for in vitro oocyte maturation comprising providing a mammalian oocyte, providing a fluid medium and a stationary support, contacting the oocyte with the stationary support in the presence of the fluid medium, and refreshing the fluid medium by simultaneously removing old fluid medium and supplying new fluid medium. In some embodiments, the stationary support comprises poly-dimethylsiloxane. In some embodiments, the stationary support comprises a poly-dimethylsiloxane-parylene-poly-dimethylsiloxane hybrid membrane. In some embodiments, the stationary support comprises channels through which said medium flows. In some embodiments, the method further comprises contacting the stationary support with a device configured to perform pulsatile fluidic operations. In some embodiments, the dynamic flow is achieved by constant peristaltic pumping. In some embodiments, the dynamic flow is achieved by reverse-exchange pumping. In some embodiments, the fluid media comprises TCM-199 with 10% fetal calf serum, 0.5 μg/ml bovine FSH, 5.0 μg/ml bovine LH, and 10 ng/ml EGF, although other suitable mediums are contemplated. In some embodiments, the method further comprises providing at least one cumulus cell. In some embodiments, the oocyte resides in a cumulus oocyte complex. In some embodiments, the refreshment of fluid medium occurs continuously. In some embodiments, the refreshment of fluid medium occurs discontinuously.

In certain embodiments, the present invention provides a method for in vitro oocyte maturation comprising providing a mammalian oocyte; culturing the oocyte in vitro in the presence of fluid medium; and subjecting the oocyte to dynamic flow during culturing wherein the dynamic flow results in refreshment of the fluid medium. In some embodiments, the dynamic flow is achieved by a device configured to perform pulsatile fluidic operations. In some embodiments, the dynamic flow is achieved by constant peristaltic pumping. In some embodiments, the dynamic flow is achieved by reverse-exchange pumping. In some embodiments, the fluid media comprises TCM-199 with 10% fetal calf serum, 0.5 μg/ml bovine FSH, 5.0 μg/ml bovine LH, and 10 ng/ml EGF, although other suitable mediums are contemplated. In some embodiments, the method further comprises providing at least one cumulus cell. In some embodiments, the oocyte resides in a cumulus oocyte complex. In some embodiments, the refreshment of fluid medium occurs continuously. In some embodiments, the refreshment of fluid medium occurs discontinuously.

Additional embodiments will be apparent to persons skilled in the relevant art based on the teachings contained herein.

### DESCRIPTION OF THE DRAWINGS

FIG. 1 shows bovine embryo development following oocyte maturation with static or dynamic flow. Embryo cleavage was assessed at 24 hours and blastocyst development was recorded at 144 hours post-fertilization. This figure represents the average of three replicates and includes standard errors of the means. Different superscripts, ^{a,b} indicate statistical significance (P<0.05) within stage of development.

FIG. 2 shows bovine embryo development from oocytes inseminated on a PDMS-platform and in traditional static culture drops. Embryo cleavage was assessed at 24 hours and blastocyst development was recorded at 144 hours post-fertilization. This figure represents the average of three replicates and includes standard errors of the means.

FIG. 3 shows development of bovine embryos cultured on a dynamic microfluidic platform and in traditional static culture drops. Embryo cleavage was assessed at 24 hours and blastocyst development was recorded at 144 hours post-fertilization. This figure represents the average of five replicates and includes standard errors of the means. Different superscripts, ^{a,b} indicate statistical significance (P<0.05) and different superscripts, ^{c,d} indicate statistical significance (P<0.001) within stage of development.

FIG. 4 shows devices used in some embodiments of the present invention. (a) PDMS-based microfluidic chip with a funnel shaped well containing bovine embryos (upper right) placed over a Braille pin actuator. The lower right shows a side view of the microfluidic chip held in place with two plastic wing nuts. (b) A schematic drawing of the microfluidic chip consisting of a center well for oocyte or embryo culture; on the left hand side illustration of an inlet and outlet reservoir for media exchange. (c) A top view of a microfluidic chip schematic drawing. Arrows indicate direction of media flow from the inlet to the outlet reservoir. This microfluidic network circulates and provides fresh media to oocytes and embryos during the culture period.

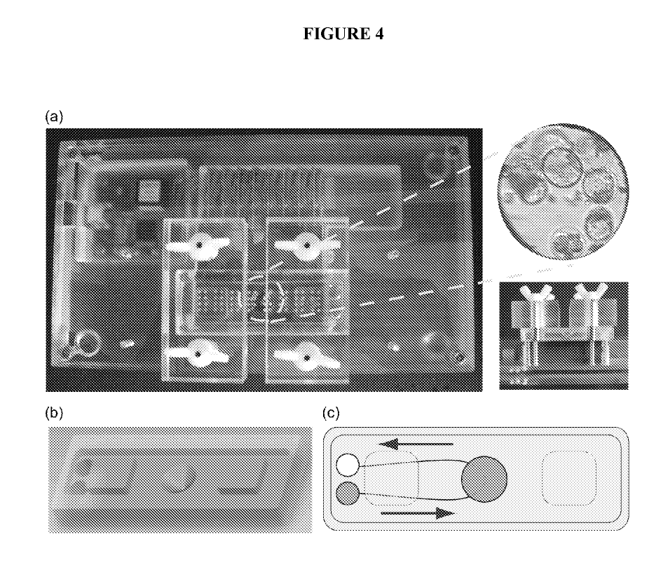

FIG. 5 shows a methodological flowchart for experiments conducted during the course of developing some embodiments of the present invention.

FIG. 6 shows developmental stages achieved for bovine oocytes matured under static in vitro maturation (static IVM) or dynamic in vitro maturation (dynamic IVM) conditions.

FIG. 7 shows exemplary O₂ diffusion steps occurring during static in vitro oocyte maturation that influence the oocyte point-of-contact oxygen concentration. The steps include the concentration of gaseous O₂ in the chamber, dissolution of O₂ in oil, dissolution of O₂ in media, utilization of O₂ by cumulus cells, and utilization of O₂ by the oocyte.

### DEFINITIONS

To facilitate an understanding of the present invention, a number of terms and phrases are defined below:

The term “sample” is used in its broadest sense. On the one hand it is meant to include a specimen or culture. On the other hand, it is meant to include both biological and environmental samples. A sample may include a specimen of synthetic origin.

As used herein, the term “cell” refers to any eukaryotic or prokaryotic cell (e.g., bacterial cells such as E. coli, yeast cells, mammalian cells, avian cells, amphibian cells, plant cells, fish cells, and insect cells), whether located in vitro or in vivo.

As used herein, the term “cell culture” refers to any in vitro culture of cells. Included within this term are continuous cell lines (e.g., with an immortal phenotype), primary cell cultures, transformed cell lines, finite cell lines (e.g., non-transformed cells), and any other cell population maintained in vitro.

As used, the term “eukaryote” refers to organisms distinguishable from “prokaryotes.” It is intended that the term encompass all organisms with cells that exhibit the usual characteristics of eukaryotes, such as the presence of a true nucleus bounded by a nuclear membrane, within which lie the chromosomes, the presence of membrane-bound organelles, and other characteristics commonly observed in eukaryotic organisms. Thus, the term includes, but is not limited to such organisms as fungi, protozoa, and animals (e.g., humans).

As used herein, the term “in vitro” refers to an artificial environment and to processes or reactions that occur within an artificial environment. In vitro environments can consist of, but are not limited to, test tubes and cell culture. The term “in vivo” refers to the natural environment (e.g., an animal or a cell) and to processes or reaction that occur within a natural environment.

As used herein, the term “oocyte maturation” refers to biochemical events that prepare an oocyte for fertilization. Such processes may include but are not limited to the completion of meiosis II. The term “oocyte nuclear maturation” specifically refers to such completion of meiosis II. The term “oocyte cytoplasmic maturation” specifically refers to cytoplasmic events that occur to instill upon the oocyte a capacity to complete nuclear maturation, insemination, and/or early embryogenesis. Oocyte cytoplasmic maturation events may include but are not limited to accumulation of mRNA, proteins, substrates, and nutrients that are required to achieve the oocyte developmental competence that fosters embryonic developmental competence.

As used herein, the term “blastocyst” refers to a thin-walled hollow structure in early embryonic development that includes a cluster of cells called the inner cell mass from which the embryo arises.

As used herein, the term “cumulus cell” refers to a cell in the developing ovarian follicles which is in direct or close proximity to an oocyte.

As used herein, the term “cumulus-oocyte complex” refers to at least one oocyte and at least one cumulus cell in physical association with each other.

As used herein, the term “dynamic flow” refers to a state in a fluid system in which fluids experience kinetic motion through the system. In some embodiments, the system is open (as opposed to contained).

As used herein, the term “medium” or “fluid medium” refers to any fluid within a system. In some embodiments, the medium or fluid medium is compatible with cell culture (e.g., supports cell viability; supports cell growth; supports cell development; does not cause toxicity or lethality to a cell).

As used herein, the term “channel” or “relief channel” refers to a structural feature. In some embodiments, a channel may comprise a three-dimensional protrusion into the surface of a material. In some embodiments, a channel is elongated in shape (e.g., rectangular prism). In some embodiments, the channel is capable of holding or transmitting fluid.

### DETAILED DESCRIPTION OF THE INVENTION

Advancements in the in vitro production of embryos (IVP) have primarily been achieved through modification of culture media composition. Culture media have been designed to closely mimic the nutrient/atmospheric environment of the female reproductive tract. However, little has been done in terms of the physical tools used in assisted reproduction. As a result, the largest advancements made in embryo culture over the past decade have been the introduction of sequential embryo culture systems which require an abrupt transfer of embryos from one media to the next. These media support embryo development not only by modification of medium components at specific time points, but also by removal of harmful waste products that build up during embryo culture.

While there have been many advances in IVP in regard to efficiency and embryo quality, these embryos are still sub-optimal compared to their in vivo counterparts (Bertolini et al. (2002) Mol. Reprod. Dev. 63:318-328; Holm et al. (2002) Reproduction 123:553-565; Khurana et al. (2000) Biol. Reprod. 62:847-856; Lonergan et al. (2003) Reproduction 126:337-346; each herein incorporated by reference in its entirety). Differences in quality of embryos produced in vitro versus in vivo result from stresses imposed on oocytes/embryos during the IVP procedure. These stresses begin as early as oocyte retrieval, when even a small reduction in handling temperature affects ability of oocytes to survive (Ravindranatha et al. (2003) Reprod. Domest. Anim. 38:21-26; herein incorporated by reference in its entirety). Additionally, IVP entails as many as 20 washes and culture drop transfers, each with the potential to impose stress (changes in pH, osmolarity, and temperature) on gametes and embryos (Xie et al. (2006) Biol. Reprod. 75:45-55; each herein incorporated by reference in its entirety). Furthermore, human factors have potential to introduce oocyte/embryo loss at each handling step. Therefore, increased attention needs to focus on improving the mechanical platform on which IVP occurs.

Recent advancements in nanotechnology and miniaturization of bio-analytical procedures have provided scientists with a powerful platform on which to study biological systems. One example of these emerging technologies is microfluidics. Microfluidics deals with the behavior, specific control and manipulation of microliter and nanoliter volumes of fluid and allows for the construction of culture systems on a size scale similar to the characteristic size (diameter) of biological objects and their local in vivo environment. Advancements in microfabrication (e.g., soft lithography) have made it possible to easily construct any desired micro-structure (Anderson et al. (2000) Anal. Chem. 72:3158-3164; Unger et al. (2000) Science 288:113-116; each herein incorporated by reference in its entirety).

Micro-scale platforms have been developed for several steps of IVP with varying results in mice and pigs. In 2001, Walters and colleagues presented preliminary data demonstrating that in vitro maturation (IVM) of porcine oocytes on microfluidic chips under static conditions resulted in similar rates of nuclear maturation as those matured under standard culture conditions (Walters et al. (2001) Theriogenology 55:497; herein incorporated by reference in its entirety) Hester and colleagues demonstrated improved rates of porcine embryo cleavage from oocytes matured on chip under static conditions (Hester et al. (2002) Theriogenology 57:723; herein incorporated by reference in its entirety).

The next step in IVP following IVM is in vitro fertilization (IVF). In 2002, Clark and coworkers investigated the ability of a PDMS/borosilicate microchannel to support porcine IVF compared with traditional porcine IVF methods (Clark et al. (2002) Biol. Reprod. 66:528; herein incorporated by reference in its entirety). No statistical differences were reported in percent fertilization or early embryo cleavage between the two fertilization systems. However, fertilization was improved when a microchannel designed to recapitulate function and structure of the oviduct was developed (Clark et al. (2005) Lab Chip 5:1229-1232; herein incorporated by reference in its entirety). Suh and colleagues demonstrated that mouse IVF can be conducted quickly and successfully within PDMS-based microfluidic channels (Suh et al. (2006) Hum. Reprod. 21:477-483; herein incorporated by reference in its entirety). Furthermore, fertilization rates were significantly increased in microchannels, compared to conventional means of insemination, when the concentration of sperm used for insemination was reduced (Suh et al. (2006) Hum. Reprod. 21:477-483; herein incorporated by reference in its entirety).

The final step in the IVP process is embryo culture and development. The embryo culture environment has been the most extensively studied and has been the main emphasis of improving IVP. Hickman and colleagues demonstrated significantly higher percentages of in vivo-derived 2-cell mouse embryos cultured in microfluidic channels reaching the morula, blastocyst and hatched blastocyst stage compared to microdrop cultured controls (Hickman et al. (2002) Comp. Med. 52:122-126; herein incorporated by reference in its entirety). Furthermore, culture of in vivo-derived 4-cell porcine embryos in PDMS/borosilicate microchannels resulted in control level blastocyst development and subsequent live births (Walters et al. (2003) Theriogenology 59:353; herein incorporated by reference in its entirety).

Cabrera and coworkers demonstrated an advantage of culturing mouse embryos on a microfluidic platform with dynamic media flow (Cabrera et al. (2006) Fertility and Sterility 86:S43; herein incorporated by reference in its entirety). These experiments utilized a computer-controlled, integrated microfluidic system with on-chip pumps and valves, powered by individually actuated Braille pins to pump fresh media through a series of elastomeric capillaries (Gu et al. (2004) PNAS 101:15861-15866; herein incorporated by reference in its entirety). Embryos cultured under dynamic conditions had significantly more cells than those cultured under static conditions. Furthermore, blastocyst cell counts following microfluidic dynamic embryo culture more closely mirrored results obtained from their in vivo counterparts (Cabrera et al. (2006) Fertility and Sterility 86:S43; herein incorporated by reference in its entirety),

Development of new culture platforms and methods that mimic in vivo environments are critical for optimizing quality of embryos produced in vitro. In experiments conducted during the course of developing some embodiments of the present invention (see, e.g., Example 1), oocytes matured under dynamic conditions showed similar rates of nuclear maturation compared to those cultured under static conditions. However, significantly more embryos produced from oocytes matured under dynamic conditions reached the blastocyst stage than those from oocytes cultured under static maturation conditions. These results show that dynamic culture better supports oocyte cytoplasmic maturation and subsequent embryonic developmental competence when compared to conventional static culture.

Full cytoplasmic maturation is associated with the reorganization of many organelles and synthesis of reducing agents, all of which are necessary for normal fertilization (Sutovsky et al. (1997) Biol. Reprod. 56:1503-1512; herein incorporated by reference in its entirety). One important component of cytoplasmic maturation that can be greatly affected by the presence of ammonium and other in vitro-induced waste products is oxidative metabolism. During bovine IVM, rapidly expanding cumulus cells surrounding the oocyte are metabolically active and involved in transporting nutrients to the maturing oocyte. During this active metabolic period, waste products accumulate in the culture environment. Exposure of granulosa cells to ammonium ions inhibits in vitro growth and metabolism of bovine granulosa cells, and negatively influences subsequent embryo development (Rooke et al. (2004) Anim. Reprod. Sci. 84:53-71; herein incorporated by reference in its entirety). While the present invention is not limited to any particular mechanism, and an understanding of the mechanism is not necessary to practice the present invention, it is contemplated that the increased blastocyst development observed in the dynamic IVM treatment is a result of continuous waste removal and replenishment of fresh media to the maturing oocytes.

Additional experiments conducted during the course of developing some embodiments of the present invention investigated the use of a PDMS-based microfluidic platform on bovine IVF (see, e.g., Example 1). A static environment was maintained on chip to prevent changes in sperm concentration. There were no statistical differences in cleavage or blastocyst rates between oocytes inseminated on chip or in traditional microdrops. This experiment demonstrated the material safety of PDMS on the function and viability of both bovine sperm and oocytes. Previously, Clark et al. used a porcine model to investigate the ability of a PDMS/borosilicate microchannel to support porcine IVF compared with traditional porcine IVF methods (Clark et al. (2002) Biol. Reprod. 66:528; herein incorporated by reference in its entirety), reporting no statistical differences in percentage fertilization or early embryo cleavage between the two fertilization systems. Clark et al. later redesigned the microchannel to imitate function and structure of the oviduct (Clark et al. (2002) Biol. Reprod. 66:528; herein incorporated by reference in its entirety). Oocytes in microchannels had a significantly higher incidence of monospermic penetration as compared to oocytes fertilized in a traditional microdrop system. While the present invention is not limited to any particular mechanism, and an understanding of the mechanism is not necessary to practice the present invention, it is contemplated that increased monospermic penetration is due to reduced sperm concentration in close proximity with the porcine oocyte (Clark et al. (2005) Lab Chip 5:1229-1232; herein incorporated by reference in its entirety).

While the present invention is not limited to any particular mechanism, and an understanding of the mechanism is not necessary to practice the present invention, it is contemplated that bovine IVF on chip is improved with a dynamic flow rate suitable to maintain sperm-egg contact, while still eliminating waste products from the culture environment. In addition it is beneficial to the oocyte if the number of sperm surrounding the oocyte is greatly reduced. Only a few hundred sperm eventually reach the ampulla of the oviduct for fertilization in humans (Ahlgren (1975) Gynecol. Inves. 6:206-214; Settlage et al. (1973) Fertil. Sertil. 24:655-661; each herein incorporated by reference in its entirety). Advancements in microchannel design and microfluidics provide an alternative method of in vivo-like insemination by decreasing volume of media and numbers of sperm needed, while increasing oocyte-sperm interaction in a dynamic environment.

Additional experiments conducted during the course of developing some embodiments of the present invention compared development of bovine embryos cultured on a dynamic microfluidic chip and in standard microdrops. There were no statistical differences in cleavage rates from embryos cultured in both treatments. This finding is similar to that observed when culturing mouse embryos on a dynamic microfluidic platform (Cabrera et al. (2006) Fertil. Steril. 86:S43; herein incorporated by reference in its entirety), where there was a significantly higher rate of blastocyst development in the dynamic treatment. Additionally, there was a significant increase in the percentage of cleaved embryos that reached the blastocyst stage in the dynamic treatment group.

Cabrera and colleagues demonstrated that dynamic microfluidic embryo culture conditions enhance the development of in vitro-grown mouse embryos, resulting in embryos with cell numbers comparable to their in vivo counterparts (Cabrera et al. (2006) Fertil. Steril. 86:S43; herein incorporated by reference in its entirety). Furthermore, mouse embryos produced under dynamic conditions have lower rates of abortion and significantly higher rates of fetal development compared to statically-cultured mouse embryos (Smith et al. (2008) Fertil. Steril. 90:S1-S2; herein incorporated by reference in its entirety). While the present invention is not limited to any particular mechanism, and an understanding of the mechanism is not necessary to practice the present invention, it is contemplated that the continuous media exchange within the microfluidic channels more closely mimics the dynamic micro-environment of the oviduct and uterus. In addition, the continuous exchange of media to the site of the growing embryos prevents buildup of harmful waste products by developing embryos.

Experiments conducted during the course of developing some embodiments of the present invention showed that important procedures of bovine IVP, including oocyte maturation, insemination, and embryo culture can be performed on a PDMS-based microfluidic device. Integration of a microfluidic platform allows automation of chemical and mechanical manipulation.

### I. Devices

In some embodiments, methods of the present invention utilize microfluidics devices for performance of fluidic-logic, biochemical and industrial applications. The devices may be constructed of any suitable material. Exemplary, non-limiting examples of microfluidic devices are described below and e.g., in PCT Patent App. No. PCT/US09/55722 and U.S. patent application Ser. No. 11/833,014, each herein incorporated by reference in its entirety. In some embodiments, the devices comprise multiple segmented species-containing channels (e.g., valves), where the pressure of the species joins or segments the channels. In some embodiments, species (e.g., fluids or pressure) are used to regulate the opening or closing of the channels.

In some embodiments, devices are made by the sandwiching of three layers (e.g., poly-dimethylsiloxane (PDMS) layers). In some embodiments, the top and bottom layers contain the main network of microfluidic channels. In some embodiments, the middle layer is a thin membrane.

In some embodiments, layers are made by supplying a negative “master” and casting a castable material over the master. Castable materials include, but are not limited to, polymers, including epoxy resins, curable polyurethane elastomers, polymer solutions (e.g., solutions of acrylate polymers in methylene chloride or other solvents), curable polyorganosiloxanes, and polyorganosiloxanes which predominately bear methyl groups (e.g., polydimethylsiloxanes (“PDMS”)). Curable PDMS polymers are well known and available from many sources. Both addition curable and condensation-curable systems are available, as also are peroxide-cured systems. All these PDMS polymers have a small proportion of reactive groups which react to form crosslinks and/or cause chain extension during cure. Both one part (RTV-1) and two part (RTV-2) systems are available. Additional curable systems are preferred when biological particle viability is needed.

In some embodiments, transparent devices are desirable. Such devices may be made of glass or transparent polymers. PDMS polymers are well suited for transparent devices. A benefit of employing a polymer which is slightly elastomeric is the case of removal from the mold and the potential for providing undercut channels, which is generally not possible with hard, rigid materials. Methods of fabrication of microfluidic devices by casting of silicone polymers are well known. See, e.g. D. C. Duffy et al., “Rapid Prototyping of Microfluidic Systems in Poly(dimethylsiloxane),” Analytical Chemistry 70, 4974-4984 (1998). See also, J. R. Anderson et al., Analytical Chemistry 72, 3158-64 (2000); and M. A. Unger et al., Science 288, 113-16 (2000), each of which is herein incorporated by reference in its entirety.

In some embodiments, fluids are supplied to the device by any suitable method. Fluids may, for example, be supplied from syringes, from microtubing attached to or bonded to the inlet channels, etc.

Fluid flow may be established by any suitable method. For example, external micropumps suitable for pumping small quantities of liquids are available. Micropumps may also be provided in the device itself, driven by thermal gradients, magnetic and/or electric fields, applied pressure, etc. All these devices are known to the skilled artisan. Integration of passively-driven pumping systems and microfluidic channels has been proposed by B. H. Weigl et al., Proceedings of MicroTAS 2000, Enshede, Netherlands, pp. 299-302 (2000).

In other embodiments, fluid flow is established by a gravity flow pump, by capillary action, or by combinations of these methods. A simple gravity flow pump consists of a fluid reservoir either external or internal to the device, which contains fluid at a higher level (with respect to gravity) than the respective device outlet. Such gravity pumps have the deficiency that the hydrostatic head, and hence the flow rate, varies as the height of liquid in the reservoir drops. For many devices, a relatively constant and non-pulsing flow is desired.

To obtain constant flow, a gravity-driven pump as disclosed in published PCT application No. WO 03/008102 A1 (Jan. 18, 2002), herein incorporated by reference, may be used. In such devices, a horizontal reservoir is used in which the fluid moves horizontally, being prevented from collapsing vertically in the reservoir by surface tension and capillary forces between the liquid and reservoir walls. Since the height of liquid remains constant, there is no variation in the hydrostatic head.

Flow may also be induced by capillary action. In such a case, fluid in the respective outlet channel or reservoir will exhibit greater capillary forces with respect to its channel or reservoir walls as compared to the capillary forces in the associated device. This difference in capillary force may be brought about by several methods. For example, the walls of the outlet and inlet channels or reservoirs may have differing hydrophobicity or hydrophilicity. Alternatively, the cross-sectional area of the outlet channel or reservoir is made smaller, thus exhibiting greater capillary force.

In some embodiments, flow is facilitated by embedded capacitor valves that pump fluids in a separate channel when pressurized. This is achieved by having a series of valves in the bottom that direct a pressurized gas or liquid causing the membrane to deform and squeeze the fluid in the top channel forward. Additional control is provided by having valves in the top layer that can open sequentially.

The device of embodiments of the present invention can be constructed using any suitable method. In some embodiments, the microfluidic devices comprise a hydraulically driven actuation component and a microfluidics component. Exemplary devices may include (1) a control channel, (2) a fluidic channel, (3) a piston, and (4) an actuator (e.g., a Braille pin). The control channel may be filled with hydraulic fluid. At least a portion (e.g., the portion to be deformed) of the control and fluid channels may preferably be constructed from flexible materials. The control and fluid channels are preferably closed channels. The hydraulic fluid may preferably be an ionic liquid or other liquid that is non-volatile and has low permeability. Ionic liquids are particularly useful as hydraulic fluids because they can hold pressure within a control channel for very long times compared to other liquids because they are non-volatile and have very low permeability.

When the device is in an open configuration, the actuator may be in the “down” position such that the hydraulic fluid is not moving through the control channel. When the device is in a closed configuration, the actuator may be in the “up” position such that the hydraulic fluid flows through the control channel and pushes on the fluidic channel, thus restricting flow through the fluidic channel. The device thus may utilize such actuation to control the flow of fluid and biological materials through the fluidic channels.

In some embodiments, the device may be utilized to provide multiplexed hydraulically actuated systems. For example, in some embodiments, a single control channel may be used to control more than one fluidic channel. In some embodiments, devices and systems comprise greater than one control channel (e.g., more than 5, more than 10, more than 50 or more than 100). In some embodiments, devices and systems comprise greater than one fluidic channel (e.g., more than 5, more than 10, more than 50 or more than 100).

In some embodiments, the control channels of the device are filled with hydraulic fluid that is an ionic liquid. The use of ionic liquid provides the advantage of not evaporating or leaking like a volatile liquid or gas. The use of ionic liquid provides the further advantage over a viscous fluid of being quicker to deform and thus allowing for more rapid valving and pumping. The ionic fluid filled channels are further suitable for use with small volumes of fluid and are able to maintain pressure long term. Such devices are thus suitable for long term use. The use of hydraulics further results in a portable, small, and low cost device.

A. Construction of Devices

In some embodiments, construction of fluidic devices is preferably by soft lithography techniques as described for example by Duffy et al (Analytical Chem 70 4974-4984 1998; See also Anderson et al, Analytical Chem 72 158-64 2000 and Unger et al., Science 288 113-16 2000). Addition-curable RTV-2 silicone elastomers such as SYLGARD™ 184 Dow Corning Co can be used for this purpose. The dimensions of the various flow channels are readily determined by volume and flow rate properties etc. Channels that are designed for complete closure are preferably of a depth such that the elastomeric layer between the microchannel and the actuator can approach the bottom of the channel. Manufacturing the substrate of elastomeric material facilitates complete closure in general as does also cross-section which is rounded particularly at the furthest corners further from the actuator. The depth also depends, for example, on the extension possible for the actuators extendable protrusions. Thus channel depths may vary from a depth of less than 100 μm preferred more preferably less than 50 μm. Channel depths in the range of 10 μm-40 μm are preferred for the majority of applications but even very low channel depths (e.g., nm) are feasible and depths of 500 μm are possible with suitable actuators particularly if partial closure partial valving is sufficient.

The substrate may be of one layer or plurality of layers. The individual layers may be prepared by numerous techniques including laser ablation, plasma etching, wet chemical methods, injection molding, press molding, etc. Casting from curable silicone is most preferred, particularly when optical properties are important. Generation of the negative mold can be made by numerous methods all of which are well known to those skilled in the art. The silicone is then poured onto the mold degassed if necessary or desired and allowed to cure. Adherence of multiple layers to each other may be accomplished by conventional techniques.

A preferred method of manufacture of some devices employs preparing a master through use of negative photoresist SU-8 50 photoresist from Micro Chem Corp Newton Mass. The photoresist may be applied to glass substrate and exposed from the uncoated side through suitable mask. Since the depth of cure is dependant on factors such as length of exposure and intensity of the light source features ranging from very thin up to the depth of the photoresist may be created. The unexposed resist is removed leaving a raised pattern on the glass substrate. The curable elastomer is cast onto this master and then removed. The material properties of SU-8 photoresist and the diffuse light from an inexpensive light source can be employed to generate microstructures and channels with cross-sectional profiles that are rounded and smooth at the edges yet flat at the top, e.g., bell-shaped. Short exposures tend to produce radiused top while longer exposures tend to produce flat top with rounded corners. Longer exposures also tend to produce wider channels. These profiles are ideal for use as compressive deformation-based valves that require complete collapse of the channel structure to stop fluid flow as disclosed by Unger et al., (Science (2000) 288: 113). With such channels, Braille-type actuators produced full closure of the microchannels thus producing very useful valved microchannels. Such shapes also lend themselves to produce uniform flow fields and have good optical properties as well. In a typical procedure, a photoresist layer is exposed from the backside of the substrate through mask, for example, photoplotted film, by diffused light generated with an ultraviolet UV transilluminator. Bell-shaped cross-sections are generated due to the way in which the spherical wavefront, created by diffused light penetrates into the negative photoresist. The exposure dose dependent change in the SU-8 absorption coefficient is 3985m-1 unexposed to 9700 m-1 exposed at 365 nm limits exposure depth at the edges. The exact cross-sectional shapes and widths of the fabricated structures are determined by a combination of photomask feature size exposure 20 time/intensity resist thickness and distance between the photomask and photoresist. Although backside exposure makes features which are wider than the size defined by the photomask, and in some cases smaller in height compared to the thickness of the original photoresist coating, the change in dimensions of the transferred patterns is readily predicted from mask dimensions and exposure time. The relationship between the width of the photomask patterns and the photoresist patterns obtained is essentially the linear slope of beyond certain photomask aperture size. This linear relationship allows straightforward compensation of the aperture size on the photomask through simple subtraction of constant value. When exposure time is held constant there is a threshold aperture size below which incomplete exposure will cause the microchannel height to be lower than the original photoresist thickness. Lower exposure doses will make channels with smoother and more rounded cross-sectional profiles. Light exposure doses that are too slow or photoresist thicknesses that are too large however, are insufficient in penetrating through the photoresist, resulting in cross-sections that are thinner than the thickness of the original photoresist.

B. Actuators

In some embodiments, the pressure required to activate the hydraulic pistons of the device is supplied by an external tactile device such as are used in refreshable Braille displays. The tactile actuator contacts the active portion of the device and when energized extends and presses upon the deformable elastomer restricting or closing the feature in the active portion. Rather than close or restrict feature by being energized the tactile actuator may be manufactured in an extended position which retracts upon energizing or may be applied to the microfluidics device in an energized state closing or restricting the passage further opening the passage upon de-energizing. In some embodiments, actuators are programmable Braille display devices such as those commercially available from Telesensory as the NAVIGATOR Braille Display with GATEWAY software which directly translates screen text into Braille code. Braille displays are available from Handy Tech Blazie and Alva among other suppliers. These devices generally provide a linear array of 8-dot cells, each cell and each cell dot of which is individually programmable. Such devices are used by the visually impaired to convert row of text to Braille symbols one row at time for example to read textual message books, etc. Additional commercially available or otherwise constructed Braille devices may be used in the devices.

The microfluidic device active portions are designed such that they are positionable below respective actuatable dots or protrusions on the Braille display. However, to increase flexibility, it is possible to provide regular rectangular arrays usable with plurality of microfluidics devices. The more close the spacing and the higher the number of programmable extendable protrusions, the greater the flexibility in design of microdevices.

Addressability also follows from customary methods. Suitable Braille display devices suitable for non-integral use are available from Handy Tech Electronik GmbH Horb Germany as the Graphic Window Professional GWP having an array of 24×16 tactile pins. Pneumatic displays operated by microvalves have been disclosed by Orbital Research Inc said to reduce the cost of Braille tactile cells from $70 U.S per cell to 5-10 $/cell. Piezoelectric actuators are also usable where piezoelectric element replaces the electro-rheological fluid and electrode positioning is altered accordingly. Additional actuator devices may be used in the methods of the present invention and are known to those of skill in the art (See e.g., U.S patent publication 20070090166, herein incorporated by reference in its entirety).

### II. Device and System Uses

In some method embodiments of the present invention, a fluidic system comprising valves and channels is employed to perform a particular task (e.g., in vitro oocyte maturation). Such systems are, e.g., described in PCT Patent App. No. PCT/US09/55722 and U.S. patent application Ser. No. 11/833,014, each herein incorporated by reference in its entirety. Employment of such a system usually means that a certain number of valves need to be operated, for example, opened or closed, in a particular sequence, and possibly for different durations, in order to accomplish the desired task.

One advantage of such systems is the auto-regulatory role of the components and the ability to incorporate them in large scale both in parallel and in series. This advantage allows complex operations to be performed with little external setup or signaling. For cases where a pre-defined operation is desired without any variable decisions, this system enables all functionality to be completely encoded into the device (that is no external electrical, pneumatic, or mechanical input is required except for the source of fluid flow). This allows users with little training to operate the devices. Therefore, in some embodiments, the systems and methods described herein are amenable for use by non-microfluidic specialists in academic or industrial labs. For applications which require a specific sequence and ratio of mixing of solutions (e.g., preparation or introduction of culture media), the systems described herein can be employed automatically with the user only needing to activate the device.

However, some in vitro culture regimes require a variable input from the user to which the device then subsequently performs a particular operation. In this case, it is preferred to have some kind of logical control over the device's operations. Logical operations to be performed by reconfiguring the geometry through input signals from the user that can either open or close transistor-valves. An example would be the metering of solutions based on desired culture medium composition. The device can have several inputs for different ranges which respectively activate a separate set of components which will deliver different amounts of solutions.

The ability of the embedded fluidic circuits to provide various pulsed patterns of agents (e.g., culture media) to cells has important applications in determining optimal culture regimes, implementing temporal changes to culture regime, etc. The small size and multiplexing capabilities especially are useful for screening many different temporal patterns of dynamic flow on cells in parallel to identify ideal conditions for culture, differentiation, mechanistic evaluations.

Another advantage of such systems is their ability to compartmentalize fluids in an unpressurized state, providing a means for device memory. Therefore cell culture regimes can be conducted which provide several output solutions which can be stored and segregated from other fluids by negating any flow or diffusion of molecules which could cross-contaminate the solutions.

Such devices and systems find particular use in oocyte maturation methods of the present invention. For example, such devices and systems may be used to provide continual refreshment of soluble environment (e.g., culture media) during in vitro oocyte maturation of mammalian oocytes. While the present invention is not limited to any particular mechanism, and an understanding of the mechanism is not necessary to practice the present invention, it is contemplated that in a static soluble environment (such as conventional static microdrops), the high metabolic activity of non-gamete cells (e.g., cumulus cells) that accompany the immature oocyte causes accumulation of waste products and/or depletes substrates in the media that are necessary for oocyte growth and development. Thus, in static environments, oocyte health and subsequent embryonic developmental competence may be compromised. Thus, while the present invention is not limited to any particular mechanism, and an understanding of the mechanism is not necessary to practice the present invention, it is contemplated that a continual dynamic culture condition enables maintenance of cumulus-oocyte cellular interactions and stimulatory cross-talk while providing renewable substrates for cumulus metabolism and removing metabolic waste produced by cumulus cells.

In some embodiments, a pumping or reverse-exchange dynamic soluble environment is advantageous as oocytes mature and cumulus cells expand, raising the risk of clogging flow-channels. In some embodiments, this pumping or reverse-exchange flow is preferential to uninterrupted flow that, once clogged, would cease to refresh the soluble environment. In some embodiments, pumping nature of the flow (e.g., pumping regime) provides a developmental advantage. While the present invention is not limited to any particular mechanism, and an understanding of the mechanism is not necessary to practice the present invention, it is contemplated that dynamic flow imparted by such devices and systems impact oocyte cytoplasmic maturation and facilitate improved subsequent embryonic developmental competence. A continually refreshed soluble environment provides stable soluble medium composition in comparison to non-refreshed (e.g., static) methods. Experiments conducted during the course of developing some embodiments of the present invention showed that significantly more blastocysts were produced from oocytes matured under dynamic flow (e.g., dynamic microfluidic) conditions than from oocytes matured under static conditions, indicating higher rates of developmental competence.

### EXAMPLES

The following examples are provided in order to demonstrate and further illustrate certain preferred embodiments and aspects of the present invention and are not to be construed as limiting the scope thereof.

### Example 1

### Development of Bovine Blastocysts Following In vitro Oocyte Maturation, Fertilization and Embryo Culture on a Microfluidic Platform

### Materials and Methods

### In Vitro Oocyte Maturation

Bovine ovaries were obtained from a local abattoir and transported to the laboratory within 2 h of collection at 32-37° C. Ovaries were rinsed twice with warmed 0.9% saline. Cumulus oocyte complexes (COCs) were aspirated from antral follicles (2-10 mm in diameter) using an 18-gauge needle (Vetpharm, Sioux Center, USA). Only COCs having at least three layers of non-expanded cumulus and an even distribution of cytoplasm were selected. Oocytes were washed three times in HEPES buffered medium supplemented with 1.0% v/v PSA (100 units/ml penicillin, 100 μg/ml streptomycin, 0.25 ng/ml amphotericin, Gibco, Grand Island, N.Y.) and once in maturation medium (TCM-199). Selected COCs were matured in tissue culture medium 199 (TCM-199; Gibco, Grand Island, N.Y.), supplemented with 10% fetal calf serum (FCS; Gibco, Grand Island, N.Y.), 0.5 μg/ml bovine FSH, 5.0 μg/ml bovine LH (Sioux BCHM, Sioux Center, Iowa) and 10 ng/ml epidermal growth factor (EGF, Sigma, St Louis, Mo.). Oocytes were matured in groups of 10 in 50 nl drops of maturation medium covered with mineral oil (Irvine Scientific, Irvine, Calif.) at 39° C. in 5% CO₂ and 100% humidity. Oocytes assigned to microfluidic chips (see microfluidic chip set-up infra) were matured in groups of 10 in 50 nl drops of maturation medium covered with mineral oil (Irvine Scientific, Irvine, Calif.) at 39° C. in 5% CO₂ and 100% humidity. No oocyte/embryo selection was performed beyond this point.

### In Vitro Fertilization

At 22 h post in vitro maturation (IVM), oocytes were washed three times in warmed HEPES-BSA and once in equilibrated fertilization media (IVF-TALP supplemented with 3 mg/ml crystallized BSA, Sigma). Oocytes were transferred in groups of 10 to 50 μl drops of IVF-TALP covered with mineral oil (Irvine Scientific, Irvine, Calif.) at 39° C. in 5% CO₂ and 100% humidity. Oocytes assigned to microfluidic chips (see microfluidic chip set-up below) were cultured in groups of 10 in 50 μl drops of IVF-TALP covered with mineral oil (Irvine Scientific, Irvine, Calif.) at 39° C. in 5% CO₂ and 100% humidity. Frozen sperm from a bull of proven fertility were thawed and viable sperm isolated using a 90/45% discontinuous gradient of Isolate (Irvine Scientific, Irvine, Calif.) and Sperm-TALP supplemented with fraction V BSA (Sigma). Following isolation, the supernatant was removed and the sperm pellet was washed and centrifuged in Sperm-TALP supplemented with fraction V BSA. Sperm were counted and used for insemination at a concentration of 1×10⁶ sperm/ml of fertilization media. Penicillamine, hypotaurine and epinephrine (PHE), and heparin (5 mg/ml) were added to the fertilization drop to stimulate sperm motility and to facilitate sperm capacitation.

### Embryo Development

Presumptive zygotes were washed 3 times in HEPES-BSA and one time in culture media (KSOM+amino acids supplemented with 3mg/ml crystallized BSA) for 7 days in 50 μl drops (10 per drop) under mineral oil at a temperature of 39° C., 100% humidity and in an atmosphere of 10% CO₂, 5% O₂ and balanced N₂. Presumptive zygotes assigned to microfluidic chips (see microfluidic chip set-up infra) were cultured in groups of 10 in 50 μl drops of culture medium covered with mineral oil (Irvine Scientific, Irvine, Calif.) at 39° C. in an atmosphere of 10% CO₂, 5% O₂ and balanced N₂. Embryos were morphologically assessed for cleavage and blastocyst development and stained for total cell count at the end of the 7 day culture period.

### Cell Staining

At 22 h post IVM oocytes were removed of cumulus cells by placing COCs into hyaluronidase (0.01 μg/ml) in HEPES-BSA for 3 min followed by vortexing for 2 min. Both oocytes and blastocysts were stained with 0.1 mg/ml Hoechst 33342 DNA stain diluted in HEPES in 30 μl drops for 30 minutes, mounted on siliconized slides and squashed under a cover slip. Stage of oocyte nuclear maturation or blastocyst total cell number was measured using a fluorescent microscope (Wall et al. (1985) Biol. Reprod. 32:645-651; herein incorporated by reference in its entirety).

### Microfluidic Chip Set-Up

Chip preparation for IVM, IVF and embryo culture was identical with respect to chip manufacturing, size, sterilization, preparation, and flow rates. The microfluidic device was composed of a thick (˜8 mm) PDMS slab with microfluidic channel features, fabricated using soft lithography (Duffy et al. (1998) Analyt. Chem. 70:4974-4984; Futai et al. (2004) Adv. Mat. 16:1320-1323; each herein incorporated by reference in its entirety), attached to a PDMS-parylene-PDMS hybrid membrane (Heo et al. (2007) Anal. Chem. 79:1126-1134; herein incorporated by reference in its entirety). The thick PDMS slab with channel features was prepared by casting prepolymer (Sylgard 184, Dow-Corning) at a 1:10 curing agent-to-base ratio against positive relief features (Duffy et al. (1998) Analyt. Chem. 70:4974-4984; Futai et al. (2004) Adv. Mat. 16:1320-1323; each herein incorporated by reference in its entirety). The relief features were composed of SU-8 (MicroChem, Newton, Mass.) and fabricated on a thin glass wafer (200 μm thick) by using backside diffused-light photolithography (Futai et al. (2004) Adv. Mat. 16:1320-1323; herein incorporated by reference in its entirety). The prepolymer was then cured at 60° C. for 60 min, and holes were punched in it by a sharpened needle. The PDMS-parylene-PDMS hybrid membrane was prepared by the following stepwise procedure: spin coating PDMS onto a 4-inch silanized silicon wafer to a thickness of 100 μm, curing this layer at 120° C. for 30 min, depositing a 2.5 or 5 μm thick parylene layer using a PDS 2010 labcoater, plasma oxidizing the resulting parylene surface for 90 seconds, and spin coating another 100 μm thick layer of PDMS and curing to get a total thickness of ˜200 μm.

On-chip peristaltic pumping was performed using multiple computer-contolled, piezoelectric, moveable pins on a custom Braille display (Futai et al. (2006) Lab Chip 6:149-154; Kamotani et al. (2008) Biomaterials 29:2646-2655; each herein incorporated by reference in its entirety). This pumping motion was set at 0.1 Hz to create a constant exchange of media from the reservoir to the site of oocyte and embryo culture.

### Experiment 1

### Effect of Dynamic Culture on Oocyte Nuclear Maturation

The first experiment was designed to determine the effect of dynamic culture during oocyte maturation on the rate of oocytes reaching Metaphase II. Oocytes were randomly divided into groups of 10 in 50 pl drops of maturation media in culture dishes, microfluidic chips without dynamic flow and on microfluidic chips with dynamic media flow. At 22 h post maturation, oocytes were denuded and chromatin stained as previously described to assess stage of meiosis. Each treatment group was replicated at least 3 times.

### Experiment 2

### Effect of Dynamic IVM on Subsequent Blastocyst Development

Experiment 2 was designed to determine the effect of dynamic culture on oocyte maturation and subsequent embryo development. Oocytes were randomly allocated into groups of 10 in 50 pl drops of maturation media in culture dishes, microfluidic chips without dynamic flow and on microfluidic chips with dynamic media flow. Oocytes were inseminated and all zygotes/embryos independent of previous IVM conditions were cultured under identical conditions on static culture dishes as previously described. Development and total cell count was measured on day 7 of embryo culture. Each treatment group was replicated at least 3 times.

### Experiment 3

### Effect of in vitro Insemination of Oocytes on a PDMS-Based Microfluidic Platform

Experiment 3 was designed to determine the effect of a PDMS-based microfluidic platform on IVF of bovine oocytes. In this study, IVM oocytes were randomly allocated to either traditional static microdrops or to a static microfluidic culture chip. Static conditions were employed during insemination to maintain a consistent sperm concentration during co-incubation with oocytes. Oocytes were inseminated with equal concentrations of sperm and were cultured under identical conditions for 18 hours. Following fertilization, cumulus cells were removed and presumptive zygotes were all placed into static culture microdrops for duration of the embryo culture period. After 7 days of culture, embryo cleavage and blastocyst rates were recorded.

### Experiment 4

### Effect of Dynamic Microfluidic Embryo Culture on Blastocyst Development

Experiment 4 was designed to measure the effect of dynamic culture on bovine embryo development. Oocytes were matured and inseminated under identical conditions and presumptive zygotes were randomly transferred into groups of 10 in 50 μl drops of culture media in culture dishes or microfluidic chips with dynamic flow. Embryo development and total cell counts were measured on day 7 of embryo culture. Each treatment group was replicated at least 5 times.

### Statistical Analysis

Developmental data were analyzed using Chi-square statistics. All values given are significant at P<0.05, unless otherwise stated.

Selected COCs from bovine antral follicles were matured on microfluidic devices to examine the effect of microfluidic culture on bovine oocyte maturation rates and subsequent embryo development. In this experiment there were no statistical differences in nuclear maturation rates between bovine oocytes matured under static and dynamic conditions. Approximately 90% of all oocytes selected for maturation reached metaphase II by 22 hours culture. These results show that bovine IVM on a microfluidic platform that provides dynamic media conditions has no deleterious effect on nuclear maturation.

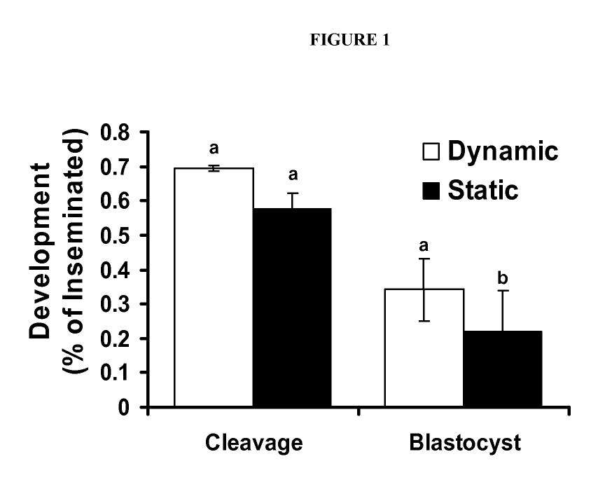

There were no statistical differences in embryo cleavage rates between embryos derived from oocytes matured in static microdrops and those matured under dynamic microfluidic conditions (58 and 69%, respectively; FIG. 1). However, significantly more blastocysts were produced from oocytes matured under dynamic microfluidic conditions than from those matured in traditional static microdrops (34 and 22%, respectively; P<0.05; FIG. 1). These results show that dynamic microfluidic culture during bovine IVM is beneficial for subsequent preimplantation embryo development competence.

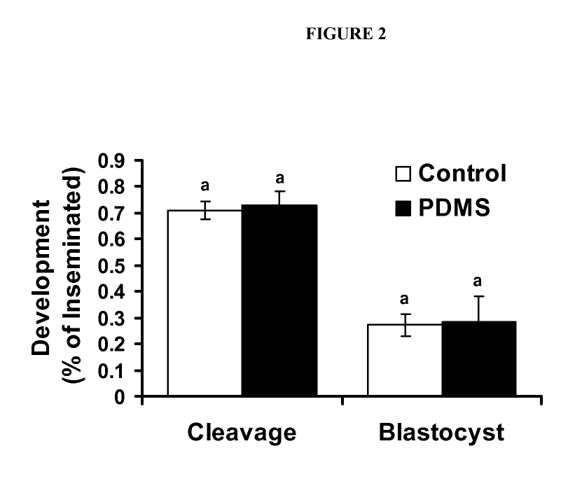

In vitro fertilization of bovine oocytes was conducted under static conditions to maintain an equal concentration of sperm. The main purpose of this experiment was to determine if a PDMS-based platform could support fertilization of bovine oocytes in vitro. There were no significant differences in the percentage of embryos that cleaved between oocytes inseminated in a microdrop and oocytes inseminated on a microfluidic chip (71 and 65%, respectively; FIG. 2). Furthermore, there were no statistical differences in blastocyst development between oocytes inseminated in a microdrop and oocytes inseminated on a microfluidic chip when the embryo development was performed under static conditions (21 and 27%, respectively; see FIG. 2). These results further demonstrate the ability to perform all aspects of IVP on a microfluidic platform. This experiment demonstrates the material safety of using a PDMS-platform on both sperm and oocyte viability and function.

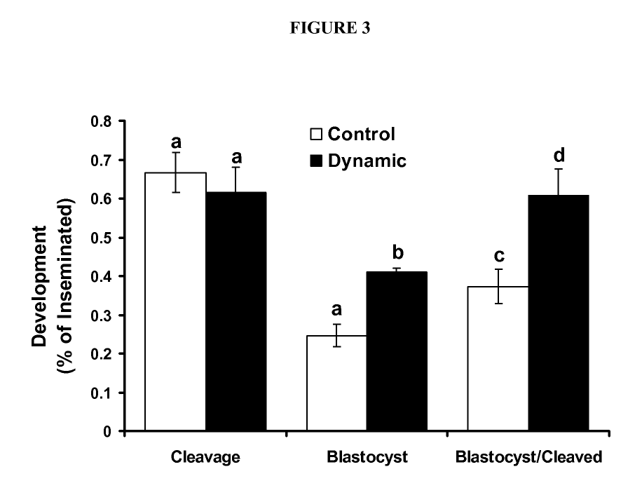

Significantly more bovine embryos from dynamic culture developed to the blastocyst stage than those cultured in static drops (35.0 vs. 21.4%, respectively; P<0.05). Moreover, the percentage of cleaved embryos developing to the blastocyst stage was dramatically increased when cultured with dynamic media flow than with conventional drops (54.0 vs. 32.3%, respectively; P<0.001; FIG. 3). Total cell counts on all blastocysts were performed at the end of the 7 day culture. The average blastocyst cell count was approximately 100 blastomeres for both treatments. The higher rate of blastocyst development in dynamic treatment further demonstrates the importance of dynamic chemical, physical, and mechanical environments on preimplantation embryo development.

### Example 2

### Oocyte Maturation, Embryo Developmental Competence, and Microfluidic Dynamic Culture

The following Example describes further experiments conducted to assess the impact of microfluidic dynamic culture conditions on mammalian (e.g., bovine) oocyte maturation.

While the mammalian oocyte spontaneously resumes meiosis and demonstrates nuclear maturation upon release from the ovarian follicle, the success of in vitro maturation of mammalian oocytes, especially human oocytes, does not support cytoplasmic maturation and subsequent efficient embryo development. In vitro maturation of human oocytes holds enormous potential in treating infertility and preserving fertility in cancer patients. It has been long recognized that the cumulus cells surrounding the oocyte are important for supporting oocyte cytoplasmic maturation and subsequent embryo development. While the oocyte has low metabolic activity, the cumulus cells have high metabolic activity. Thus the two cell types interact and are inter-dependent for oocyte developmental competence, but have different culture requirements.

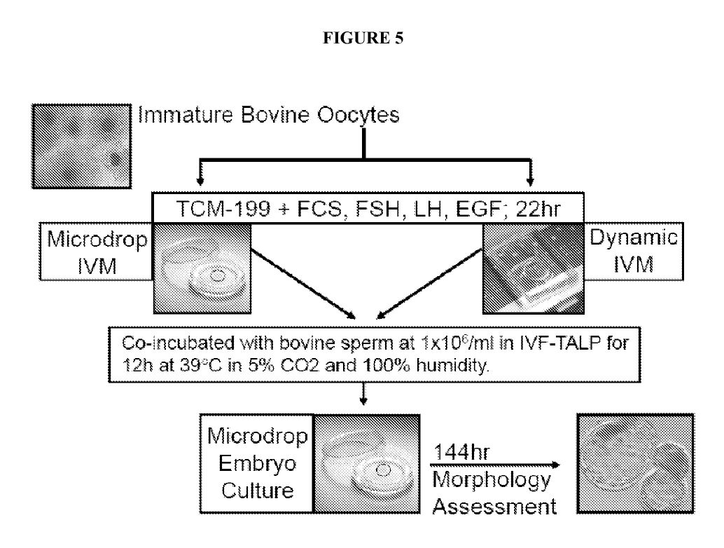

While the present invention is not limited to any particular mechanism, and an understanding of the mechanism is not necessary to practice the present invention, it is contemplated that culture of oocyte-cumulus complexes (OCCs) is improved with continual nutrient refresh and waste removal to support both normal cumulus and oocyte development. A microfluidic system was designed to act as a microcirculatory system to continually bring in new metabolic and grow substrates, slightly rock the OCC, and continually remove metabolic waste products like ammonium. Culture of bovine OCC was performed in contemporary gamete/embryo growth media with protein, hormone and growth factor addition for 22 h in microdrops (standard contemporary practice) under oil either under static conditions (microdrop under oil in petri dish) or with microfluidic dynamic media system (using same media, volume, oil overly, incubator conditions, etc). The only difference between the two treatments of OCC in vitro maturation was static versus microfluidic dynamic culture. After 22 h of in vitro maturation, oocytes were removed from OCCs, inseminated, and grown as embryos for 144 h under identical contemporary conditions (FIG. 5). The sole difference in the entire experiment was OCC in vitro maturation in either static media exposure or microfluidic dynamic media exposure.

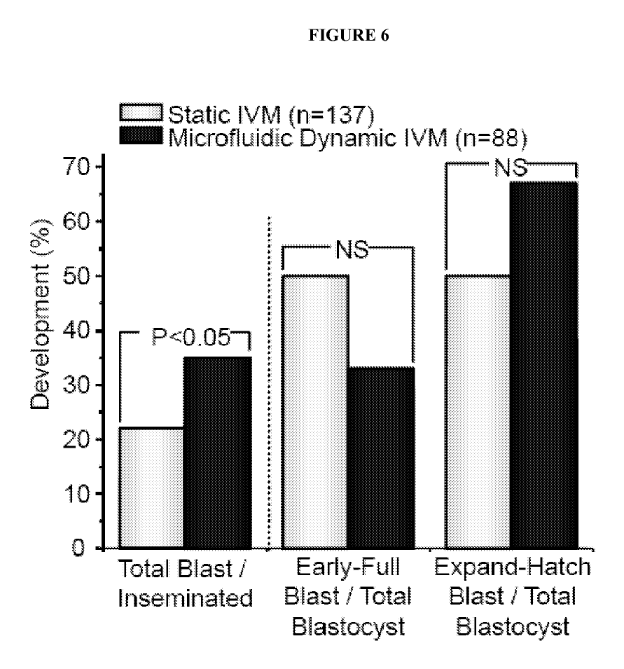

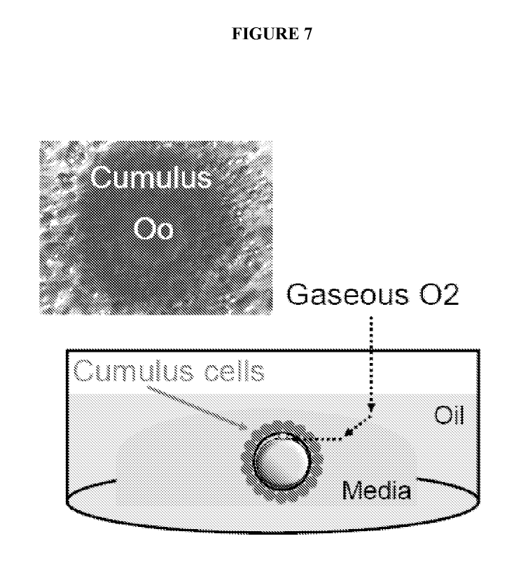

As shown in FIG. 6, no impact of treatment was observed on nuclear maturation (development to metaphase II), fertilization rate, or initial cleavage. However, if OCCs were cultured and matured in microfluidic dynamic conditions, they had significantly better embryo development to the blastocyst stage compared to OCCs cultured under standard static conditions. While the present invention is not limited to any particular mechanism, and an understanding of the mechanism is not necessary to practice the present invention, it is contemplated that the oocytes from the OCCs culture with microfluidic dynamic media exposure have significantly better cytoplasmic maturation, better embryonic developmental competence, and better embryo development compared to the contemporary static culture conditions (P<0.05). Additionally, not only was development to blastocyst more efficient, the blastocysts that formed under microfluidic dynamic culture were more advanced in their development (more expanded/hatching blastocyst) compared to standard culture. FIG. 7 shows a schematic mechanistic representation of benefit of microfluidic dymanic culture for OCC (or, by extension, any other cell culture system with two populations of cells with differential sensitivity to compounds, substrate needs, sensing of products or external mechanical signals).

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in molecular biology, in vitro fertilization, development, or related fields are intended to be within the scope of the following claims.

1. A method for in vitro oocyte maturation comprising:
a) providing a mammalian oocyte;
b) providing a fluid medium and a stationary support;
c) contacting said oocyte with said stationary support in the presence of said fluid medium; and
d) refreshing said fluid medium by simultaneously removing old fluid medium and supplying new fluid medium, resulting in production of a mature oocyte.

2. The method of claim 1, wherein said stationary support comprises poly-dimethylsiloxane.

3. The method of claim 1, wherein stationary support comprises a poly-dimethylsiloxane parylene-poly-dimethylsiloxane hybrid membrane.

4. The method of claim 1, wherein said stationary support comprises channels through which said medium flows.

5. The method of claim 1, further comprising contacting said stationary support with a device configured to perform pulsatile fluidic operations.

6. The method of claim 1, wherein said dynamic flow is achieved by constant peristaltic pumping.

7. The method of claim 1, wherein said dynamic flow is achieved by reverse-exchange pumping.

8. The method of claim 1, further comprising providing at least one cumulus cell.

9. The method of claim 1, wherein said oocyte resides in a cumulus oocyte complex.

10. The method of claim 1, wherein said refreshment of fluid medium occurs continuously.

11. The method of claim 1, wherein said refreshment of fluid medium occurs discontinuously.

12. A method for in vitro oocyte maturation comprising:
a) providing a mammalian oocyte;
b) culturing said oocyte in vitro in the presence of fluid medium; and
c) subjecting said oocyte to dynamic flow during said culturing wherein said dynamic flow results in refreshment of said fluid medium, resulting in production of a mature oocyte.

13. The method of claim 12, wherein said dynamic flow is achieved by a device configured to perform pulsatile fluidic operations.

14. The method of claim 12, wherein said dynamic flow is achieved by constant peristaltic pumping.

15. The method of claim 12, wherein said dynamic flow is achieved by reverse-exchange pumping.

16. The method of claim 12, further comprising providing at least one cumulus cell.

17. The method of claim 12, wherein said oocyte resides in a cumulus oocyte complex.

18. The method of claim 12, wherein said refreshment of fluid medium occurs continuously.

19. The method of claim 12, wherein said refreshment of fluid medium occurs discontinuously.

20. The method of claim 12, further comprising the use of said mature oocyte for in vitro fertilization to result in a fertilized cell.

21. The method of claim 20, wherein said in vitro fertilization occurs on a stationary support.

22. The method of claim 20, further comprising in vitro culture of said fertilized cell to result in a blastocyst.

23. The method of claim 22, wherein said in vitro culture of said fertilized cell occurs on a stationary support.
